(19) United States
(12) Patent Application Publication
(10) Pub. No.: US 2020/0342954 A1
(43) Pub. Date: Oct. 29, 2020

(54) Polypharmacy Side Effect Prediction With Relational Representation Learning

(71) Applicant: ACCENTURE GLOBAL SOLUTIONS LIMITED, DUBLIN (IE)

(72) Inventors: Qurrat Ul Ain, Dublin (IE); Luca Costabello, Newbridge (IE)

(73) Assignee: ACCENTURE GLOBAL SOLUTIONS LIMITED, DUBLIN (IE)

(21) Appl. No.: 16/451,709

(22) Filed: Jun. 25, 2019

Related U.S. Application Data
(60) Provisional application No. 62/838,074, filed on Apr. 24, 2019.

(51) Int. Cl. G16B 15/30; G16B 40/00; G06N 5/02

(52) U.S. Cl. CPC G16B 15/30; G06N 5/02; G16B 40/00

(57) ABSTRACT

A system adapted to receive a knowledge base, which may include drug data, human biological data, drug-drug interactions, protein-protein interactions, gene expression, protein and drug interaction data, genotypic information for cell lines, drug side effects, and disease classification labels. The system may generate a knowledge graph based on the knowledge base, and convert the knowledge graph into embeddings that include points in a k-dimensional metric space. The system may determine a medical effect weighting based on a drug combination query, and update the embeddings of the drug combination. The system may utilize a pooling method to update predicate embeddings. The system may determine polypharmacy scores for the embeddings, and rank the predicted links between a drug combination and side effects.

### CROSS-REFERENCE TO RELATED APPLICATION(S)

This application claims benefit to U.S. Provisional Patent Application No. 62/838,074, filed on Apr. 24, 2019, the entirety of which is incorporated by reference herein.

### FIELD OF THE INVENTION

The present disclosure relates in general to the fields of bioinformatics and embedding space generation, and in particular to methods and systems for predicting drug side effects of drug combinations using embedding space generated from a knowledge graph by modeling medical effect weighting and scoring predictions based on drug molecular-structure data and drug side-effect data.

### BACKGROUND

Basic techniques and equipment for machine learning, modeling data, graph embedding, and ranking drug compounds based on experimental data are known in the art. Enterprise systems have access to large volumes of information, both proprietary and public, relating to gene expression, drug interactions, molecular structures, and disease classification. Existing analytical applications and data warehousing systems have not been able to fully utilize such information. Often times, drug-related information is simply aggregated into large data warehouses without the inclusion of an added layer of relationship data connecting the information. Such aggregation of large amounts of data, without contextual or relational information, are data dumps that are not useful. Drug-related information stored in data warehouses are likely to exist in their original format, thus expending large amounts of computing resources to transform the information into searchable data in order to respond to a query.

Traditional approaches for searching drug-related data typically entail using string matching mechanisms. However, such previous approaches are limited in their ability to provide queried data. Moreover, most of the stored data is not easily searchable or available for analytics. Accordingly, conventional knowledge query systems return results that do not provide a useful picture of available data, requiring extra consumption of computing resources as knowledge queries are repeated and return inaccurate or incomplete results.

Drug-related data may be stored in different data stores depending on factors including data structure, volatility, volume, or other measurable attribute. These data stores may be designed, managed, and operated by different units within an enterprise organization. In practice, such data stores behave as data silos that are disparate, isolated, and make data less accessible across the units. More transparent and open data storage solutions are desired by pharmacological organizations to more efficiently and effectively share, access and analyze information. A multi-relational link prediction is desired to more efficiently and effectively identify side effects for drug combinations using machine-learning methods.

### BRIEF SUMMARY

The present disclosure may be embodied in various forms, including without limitation a system, method or computer-readable medium to predict the relationship or association between a drug side effect and a drug combination. In some embodiments, pre-existing drug data and human biological data may be used to generate a knowledge graph. Some embodiments of the present disclosure may include graphical representations that employ a knowledge base comprising drug data, human biological data, drug-drug interactions, protein-protein interactions, gene expression, protein and drug interaction data, genotypic information for cell lines, drug side effects, and disease classification labels. A schema for a knowledge graph may be utilized to graphically represent statements, expressed in a triples format, describing the knowledge base in order to extract sought-after information concerning the relationships between the information resourced in the knowledge base.

The graphical representation may be utilized to generate an embedding space. The embedding space may include nodes, which may represent the sets of triples that describe the relationship or predicate between a subject and an object. Accordingly, the nodes may represent embedding vectors corresponding to the structured datasets that describe the knowledge base. The inclusion of additional layers of relationship data may enable the modeling of medical effect weighting for a drug combination in order to score the prediction of the relationship between a drug combination and a drug side effect. Medical effect weighting may be utilized in a neural network, and may be selected from a combination-index database based on a drug combination query, in accordance with certain embodiments. In embodiment, the weighting of medical effect may utilize the Loewe additivity model and/or the Bliss independence model for evaluating, indexing and scoring drug combinations and their interactions.

A polypharmacy scoring function may rank the predicted links between the drug combinations and their potential side effects. The structural information of drug compounds, their associated biological data, and their effectiveness may be used to model drug combinations and determine side effect predictions. In additional to ranking the link prediction that a drug combination has a side effect, the benefits of this disclosure may include a reduction in the time spent during experimental testing by identifying side effects for certain drug combinations. Embodiments may enable a user to input a drug combination query and receive a polypharmacy score based on the medical effect weighting for the drug combination.

### BRIEF DESCRIPTION OF THE DRAWINGS

The foregoing and other objects, features, and advantages for embodiments of the present disclosure will be apparent from the following more particular description of the embodiments as illustrated in the accompanying drawings, in which reference characters refer to the same parts throughout the various views. The drawings are not necessarily to scale, emphasis instead being placed upon illustrating principles of the present disclosure.

FIG. 1 is a schematic diagram illustrating a knowledge graph schema, in accordance with certain embodiments of the present disclosure.

FIG. 2 is a block diagram illustrating an embodiment of a system for implementing polypharmacy scoring, in accordance with certain embodiments of the present disclosure.

FIG. 3 is a block diagram illustrating an embodiment of a computer architecture for a computer device for implementing polypharmacy scoring, in accordance with certain embodiments of the present disclosure.

FIG. 4 is a flow diagram illustrating the use of knowledge graph for polypharmacy scoring to identify a link prediction, in accordance with certain embodiments of the present disclosure.

FIG. 5 is a flowchart illustrating an embodiment for determining polypharmacy scores for embeddings, in accordance with certain embodiments of the present disclosure.

FIG. 6 is a flowchart illustrating an embodiment for generating candidate statements and probability metrics for the association of side effects with drug combinations to determine polypharmacy scores, in accordance with certain embodiments of the present disclosure.

FIG. 7 is a flowchart illustrating an embodiment for generating polypharmacy scores based on a knowledge graph, a drug combination query, combination-index data, in accordance with certain embodiments of the present disclosure.

### DETAILED DESCRIPTION OF EMBODIMENTS OF THE INVENTION

Reference will now be made in detail to the embodiments of the present disclosure, examples of which are illustrated in the accompanying drawings.

The present disclosure may be embodied in various forms, including a product, a system, a method and a computer readable medium for polypharmacy scoring via knowledge graph embeddings based on drug molecular-structure data and drug side-effect data for a drug combination in order to predict side effect. A knowledge base 1 of drug-related data and associated relationships may be represented in a meaningful and understandable manner via knowledge graphs 2, in accordance with certain embodiments. The model for a knowledge graph 2 may be defined by a schema or layout 3 that describes the data structures 4 and their relationships 5, which may be represented by nodes 4′ and edges 5′ in the knowledge graph 2. The knowledge graph 2 may present complex and innovative graphical structures that represent the relevant information in response to a query. In an embodiment, the knowledge graph 2 may represent the knowledge base 1 via graphical representations that correspond to structured data points or entities 4 (represented by nodes 4′), relationships 5 (represented by edges 5′), and attributes 6 (represented by node properties or edge properties 6′) with semantic meaning. The graphically represented data offered by the knowledge graph 2 may provide semantic meaning for the knowledge base 1 by modeling the data via ontology, such as a schema 3.

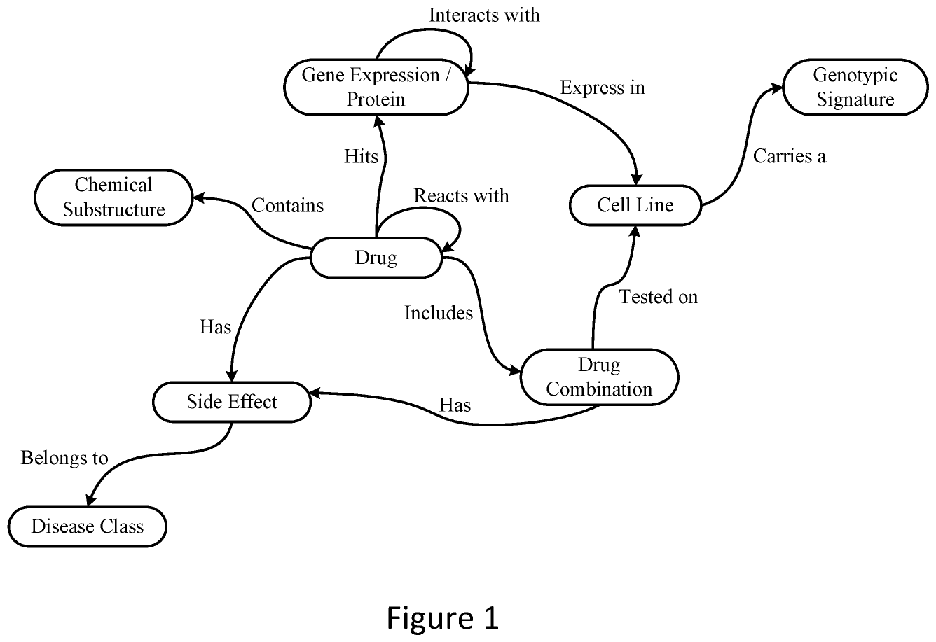

FIG. 1 illustrates a knowledge graph schema 3 within the scope of drug side effects and associated information, in accordance with certain embodiments of the present disclosure. The schema 3 may describe specific concepts or categories, e.g. classes in object-oriented data models or programming languages. The schema 3 may provide a manner to express statements 17 about the resources or knowledge base 1 using specific properties that describe the knowledge base 1. For example, a Resource Description Framework (RDF) may provide a data model that represents the knowledge base 1 in expressions of the form subject-predicate-object, known as triples 7. The subject may denote the resource, and the predicate may denote traits or aspects of the resource and express a relationship 5 between the subject and the object. For example, the statement “the drug combination has the side effect” 17 in the RDF model may be represented as the triple 7: a subject denoting the “Drug Combination”; a predicate denoting “Has”; and an object denoting “Side Effect.”

A collection of RDF statements 17 may be represented by a knowledge graph 2 having labeled vertices or nodes 4′, and labeled edges that may have a distinguished direction. Such knowledge graphs 2 may be complex multigraphs that include multiple adjacencies or edges, including self-loops, between multiple vertices or nodes 4′. For example, a side effect may be associated with a single drug or a drug combination. A data structure 4 may have properties 6, which may be represented as subjects or objects of triples 7, as defined by the knowledge graph schema 3.

Table 1 shows an example RDF-based layout or schema 3 that captures a triple 7 including a subject, predicate (e.g., relationship 5), and object, in accordance with certain embodiments of the present disclosure.

TABLE 1

| Subject | Predicate | Object |
|---|---|---|
| Drug | Contains | Chemical Structure |
| Drug | Hits | Gene/Protein |
| Drug | Reacts With | Drug |
| Drug | Has | Side Effect |
| Drug Combination | Includes | Drug |
| Drug Combination | Has | Side Effect |
| Side Effect | Belongs To | Disease Class |
| Drug Combination | Test On | Cell Line |
| Gene/Protein | Expressed In | Cell Line |
| Gene/Protein | Interacts With | Gene/Protein |
| Cell Line | Carries A | Genotypic Signature |

Each of the triples 7 describes how the subject relates to the object. In the exemplary RDF-based layout, the “Side Effects” of inflammation and vomiting may “Belong To” certain “Disease Classes”. In an embodiment, the “Drug” subject may correspond to a specific drug compound 8, and the “Chemical Structure” object may comprise the simplified molecular-input line-entry system (SMILES) strings or the structural information for that drug compound 8. A “Drug Combination” subject, corresponding to a specific drug combination 9, may comprise one or more “Drug” objects. A “Cell Line” may comprise a particular biological unit collected from a patient, such as biological data concerning a patient. A genotypic signature may comprise genetic makeup data for a patient with a particular disease. A “Protein,” or the “Gene” used to synthesize that protein, may be “Expressed In” a “Cell Line.” The disclosed triples 7 and schemas 3 may provide the advantage of intelligently integrating the knowledge base 1 into structured models and datasets to enable deep learning processes and systems that provide an improved analysis of side effects 10 associated with certain combinations 9 of drug compounds 8.

The RDF format differs from relational database tables, whose relations are pre-defined at design time and are implicit across the rows and columns of a table. Instead, RDF relationships 5 are explicitly stored as properties. In a graph-based representation, these properties may be associated with the edges 5′ that connect vertices 4′ in the graph 2. The explicit storage of these relationships 5 provides the context for interpretation of the parameters or attributes 6 for the drug compounds 8. Further, storage of the relationship 5 in addition to the parameter 6 allows for alteration of the relationship 5 without altering the parameter 6, and vice versa. The independent adjustment of these factors allows the logic to support extensions or changes to the knowledge base 1 via an adjustment of parameters 6 or relationships 5, rather than using a single degree of freedom. In an embodiment, these storage formats and datasets may benefit the identification and analysis of side effects 10 for drug combinations 9.

The present disclosure may utilize the enhanced level of structured data offered by knowledge graphs 2 to identify new and useful combinations of information extracted from the existing information received from the knowledge base 1. To accomplish this result, the present disclosure describes embedding techniques for translating the knowledge graph 2 to a plot of nodes 12′ within an embedding space 11 that represent embedding vectors 12. These techniques may further include knowledge graph enhancements that comprise selecting an area of interest within the embedding space 11, identifying empty areas within the area of interest in the embedding space 11, identifying a center node 12′ from the empty areas, and reconstructing relationships (i.e., edges or connections) of new nodes 12′ that represent the center nodes 12′. The new node 12′ may correspond to a new triple 7 not represented in the knowledge graph 2, creating an updated or sequential knowledge graph 2. Those new nodes 12′ are depictions of the center nodes 12′ from the embedding space 11, and may represent new drug combinations 9 not expressed in the original knowledge graph 2.

The features described herein are applicable to knowledge graphs 2 of data representing various fields of medicine, and may represent information within a specific field such as drug combinations 9 and drug side effects 10 for a particular field of diseases 13. In the example of the knowledge graph enhancement representing drug combination data, the new nodes 4′ in the updated knowledge graph 2 may include a chemical formulation for an existing drug combination 9, that has been updated with new added drug compounds 8. However, the knowledge graph 2 is initially generated from information received in the knowledge base 1. Constructing the updated knowledge graph 2 may include at least two steps. First, a graph schema 3 is obtained for the knowledge graph 2 and a refinement is applied as the knowledge graph 2 is being generated. This defines the types of vertices 4′ and edges 5′ that are generated into the knowledge graph 2. Second, the knowledge graph 2 is populated with information by ingesting the knowledge base 1 from one or more data sources, and applying one or more knowledge extraction techniques (e.g., natural language processing (NLP), schema mapping, computer visions, or the like) to create the vertices 4′ and edges 5′ in the knowledge graph 2.

Each data source may create its own data processing pipeline for extracting data to include into the knowledge graph 2 being constructed. The resulting knowledge graph 2 provides a specific format of structured data where each node 4′ includes information, and each connecting edge 5′ represents a relationship 5 between nodes 4′. For example, FIG. 1 illustrates a knowledge graph schema 3 including information pertaining to known drug compounds 8 and their side effects 10, where each node 4′ includes information and each edge 5′ represents a relationship 5 between the information included in the nodes 4′. To provide additional context of the technical field, including network or knowledge graph training, embedding space generation and link prediction, the entire contents of U.S. Pat. No. 10,157,226, issued on Dec. 18, 2018, U.S. Pat. No. 10,205,734, issued on Feb. 12, 2019, and U.S. Pat. No. 10,262,079, issued on Apr. 16, 2019, are hereby incorporated by reference.

In accordance with certain embodiments, an embedding space 11 may be generated from a knowledge graph 2. Graph embeddings 12 may be used to fully analyze the data for several reasons. Machine learning on graphs is limited in comparison with approaches used in vector spaces 11. Embedding spaces 11 are compressed representations of the data, which pack node properties in an embedding vector 12, that are more practical to use in equation operations than an adjacency matrix that describes connections between nodes 4′ in a graph. Further, vector operations are simpler and faster than comparable operations on graphs. Many embedding approaches are known in the art, including factorization approaches, random walk approaches, deep approaches, structural deep network embedding (SDNE) approaches, vertex embedding approaches, and graph embedding approaches. In an embodiment, the approach may comprise: sampling and relabeling sub-graphs around the selected node 4′; training the model to maximize the probability of predicting a sub-graph that exists in the graph on the input; and computing embedding spaces 11 based on a hidden layer. Accordingly, technical improvements are realized when a computing device structures information into embedding spaces 11 based on knowledge graphs 2 and runs search queries 14 on the embedding spaces 11, which specifically result in the retrieval of more relevant and accurate information, in a shorter amount of time. Furthermore, calculations may be performed to predict the relationship 5 between a drug side effect 10 and a drug combination 9, and rank the link predictions 15 using polypharmacy scoring functions 16.

In some implementations, the embedding vectors 12 may include points 12′ in a k-dimensional embedding space 11, and may provide latent semantic representations for structured knowledge in the sequential knowledge graph 2. In some implementations, the embeddings 12 may enable direct explicit relational inferences among entities via simple calculation of embedding vectors 12, and may be effective at highlighting key concepts underlying sophisticated data. In some implementations, a loss function may be minimized in order to learn optimal parameters that best discriminate positive statements from negative statements. In such implementations, the loss function may include a function that maps a statement 17 onto a real number (e.g., a metric representation 26) that represents the likelihood of that statement 17 to be true. In such implementations, the loss function may include a pairwise margin-based loss function, a negative log-likelihood loss function, and/or the like. In some implementations, the activity signature platform may assign scores to statements of the sequential knowledge graph 2 in order to aid the loss function in determining how well the sequential knowledge graph 2 tells positive statements from negative statements.

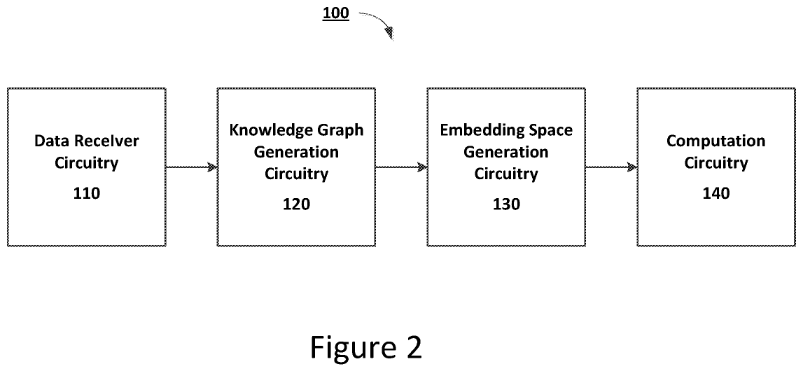

FIG. 2 illustrates an embodiment of a system 100 for implementing polypharmacy scoring. The circuitry described herein may include the hardware, software, middleware, application program interfaces (APIs), and/or other components for implementing the corresponding features of the circuitry. Initially, a data receiver circuitry 110 may be configured to receive drug molecular-structure data 29 and drug side-effect data 30. A knowledge graph generation circuitry 120 may be configured to generate a graphically represented data structure model based on the received dataset, e.g. the knowledge base 1. The knowledge graph generation circuitry 120 may construct a knowledge graph 2 from the received information 1 that is mapped to a predefined graph schema 3, in accordance with certain embodiments. The resulting graphical representation provides a specific format of structured data where each connecting edge 5′ between two nodes 4′ represents a relationship 5 between two entities 4. The knowledge graph generation circuitry 120 may further train the knowledge graph 2, in accordance with certain embodiments.

The polypharmacy scoring system 100 may further include an embedding space generation circuitry 130 that may be configured to generate embedding spaces 11 based on knowledge graphs 2. The embedding space generation circuitry 130 may convert the data 4 and relationships 5 represented in the knowledge graph 2 into a plot of nodes 12′ within an embedding space 11. The generated embedding space 11 may include vector nodes 12′ (e.g., representations of embedding vectors 12 that may correspond to triples 7) representing the structured information included in the knowledge graph 2.

In some embodiments, the system 100 may include a computation circuitry 140 for implementing computations within the embedding space 11. For example, the computation circuitry 140 may be configured to: determine a plurality of candidate statements 17; determine a medical effect weighting 18 from the combination index (CI) 19 based on the drug combination query 14; determine a polypharmacy score 20 for each candidate statement 17 based on the drug combination query 14 and based on the medical effect weighting 18 using the embedding space 11 of the knowledge graph 2; and/or, rank the predicted links 15 between the drug combinations 9 and their potential side effects 10. The computation circuitry 140 may enable the modeling of medical effect weighting 18 for a drug combination 9 in order to score the prediction 15 of the relationship 5 between a drug combination 9 and a drug side effect 10.

In some embodiments, the candidate statements 17 may represent associations 27 between a plurality of side effects 10 and a plurality of drug combinations 9. A metric representation 26 may be determined for each candidate statement 17. Each metric representation 26 may comprise the probability of an association 27 between one of the side effects 10 and one of the drug combinations 9. The metric representations 26 of the candidate statements 17 may be based on a medical effect weighting 18 of the embeddings 12. The medical effect weighting 18 may be based on a drug combination query 14. A polypharmacy score 20 may be determined for each candidate statement 17. The scores 20 may be based on the metric representations 26. In certain embodiments, the metric representations 26 of the candidate statements 17 may be ranked, and the polypharmacy scores 20 may be based on the metric representations ranking 28.

In addition, the computation circuitry 140 may identify gap regions 21 within the region of interest 22, and compute Max-Min Multi-dimensional computations to determine a center 23 for the gap regions 21 within the region of interest 22. The computation circuitry 140 is further configured to consider that center 23 to be a center node 12′ in the embedding space 11 that represents a newly discovered drug combination 9 that was not present in the original knowledge graph 2. This may be technically implemented by generating a new node 12′ within the embedding space 11 at the determined center 23 having the attributes 6 of the newly discovered combination 9. Overall, executing the polypharmacy scoring process provides improvements to the computing capabilities of a computer device executing the process by reducing the search space and by allowing for more efficient data analysis to analyze large amounts of data in a shorter amount of time.

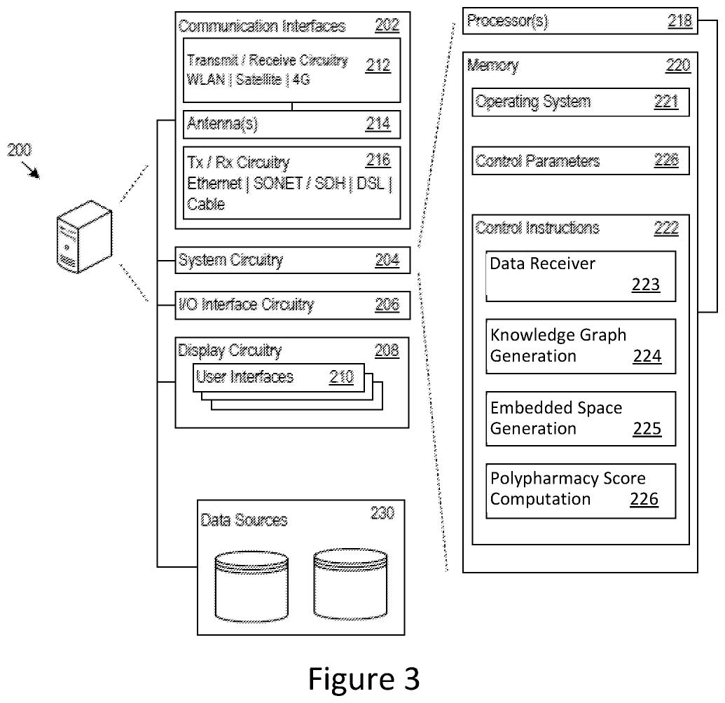

FIG. 3 illustrates a computer architecture of a computer device 200 on which the features of the polypharmacy scoring system 100 may be executed. The computer device 200 includes communication interfaces 202, system circuitry 204, input/output (I/O) interface circuitry 206, and display circuitry 208. The graphical user interfaces (GUIs) 210 displayed by the display circuitry 208 may be representative of GUIs generated by the system 100 to present a query to an enterprise application or end user, requesting information on a compound 8 to be replace and/or compound attributes 6 desired to be satisfied by a candidate discovery compound 8. The graphical user interfaces (GUIs) 210 displayed by the display circuitry 208 may also be representative of GUIs generated by the system 100 to receive query inputs identifying the drug compound 8 to be replace and/or compound attributes 6 desired to be satisfied by a candidate discovery compound 8. The GUIs 210 may be displayed locally using the display circuitry 208, or for remote visualization, e.g., as HTML, JavaScript, audio, and video output for a web browser running on a local or remote machine. Among other interface features, the GUIs 210 may further render displays of any new formulations resulting from the replacement of compounds(s) 8 with discovery compound(s) 8 selected from the processes described herein.

The GUIs 210 and the I/O interface circuitry 206 may include touch sensitive displays, voice or facial recognition inputs, buttons, switches, speakers and other user interface elements. Additional examples of the I/O interface circuitry 206 includes microphones, video and still image cameras, headset and microphone input/output jacks, Universal Serial Bus (USB) connectors, memory card slots, and other types of inputs. The I/O interface circuitry 206 may further include magnetic or optical media interfaces (e.g., a CDROM or DVD drive), serial and parallel bus interfaces, and keyboard and mouse interfaces.

The communication interfaces 202 may include wireless transmitters and receivers (herein, “transceivers”) 212 and any antennas 214 used by the transmit-and-receive circuitry of the transceivers 212. The transceivers 212 and antennas 214 may support WiFi network communications, for instance, under any version of IEEE 802.11, e.g., 802.11n or 802.11ac, or other wireless protocols such as Bluetooth, Wi-Fi, WLAN, cellular (4G, LTE/A). The communication interfaces 202 may also include serial interfaces, such as universal serial bus (USB), serial ATA, IEEE 1394, lighting port, I²C, slimBus, or other serial interfaces. The communication interfaces 202 may also include wireline transceivers 216 to support wired communication protocols. The wireline transceivers 216 may provide physical layer interfaces for any of a wide range of communication protocols, such as any type of Ethernet, Gigabit Ethernet, optical networking protocols, data over cable service interface specification (DOCSIS), digital subscriber line (DSL), Synchronous Optical Network (SONET), or other protocol.

The system circuitry 204 may include any combination of hardware, software, firmware, APIs, and/or other circuitry. The system circuitry 204 may be implemented, for example, with one or more systems on a chip (SoC), application specific integrated circuits (ASIC), microprocessors, discrete analog and digital circuits, and other circuitry. The system circuitry 204 may implement any desired functionality of the system 100. As just one example, the system circuitry 204 may include one or more instruction processor 218 and memory 220.

The memory 220 stores, for example, control instructions 222 for executing the features of the system 100, as well as an operating system 221. In one implementation, the processor 218 executes the control instructions 222 and the operating system 221 to carry out any desired functionality for the polypharmacy scoring system 100, including those attributed to data receiver 223 (e.g., relating to the data receiver circuitry 110), knowledge graph generation 224 (e.g., relating to the knowledge graph generation circuitry 120), embedding space generation 225 (e.g., relating to the embedding space generation circuitry 130), and/or polypharmacy score computation 226 (e.g., relating to the computation circuitry 140). The control parameters 227 provide and specify configuration and operating options for the control instructions 222, operating system 221, and other functionality of the computer device 200.

The computer device 200 may further include various data sources 230. Each of the databases that are included in the data sources 230 may be accessed by the system 100 to obtain data for consideration during any one or more of the processes described herein. For example, the data receiver circuitry 110 may access the data sources 230 to obtain the information for generating the knowledge graph 2 and the embedding space 11. In an embodiment, a data receiver circuitry 110 may be configured to receive a knowledge graph 2.

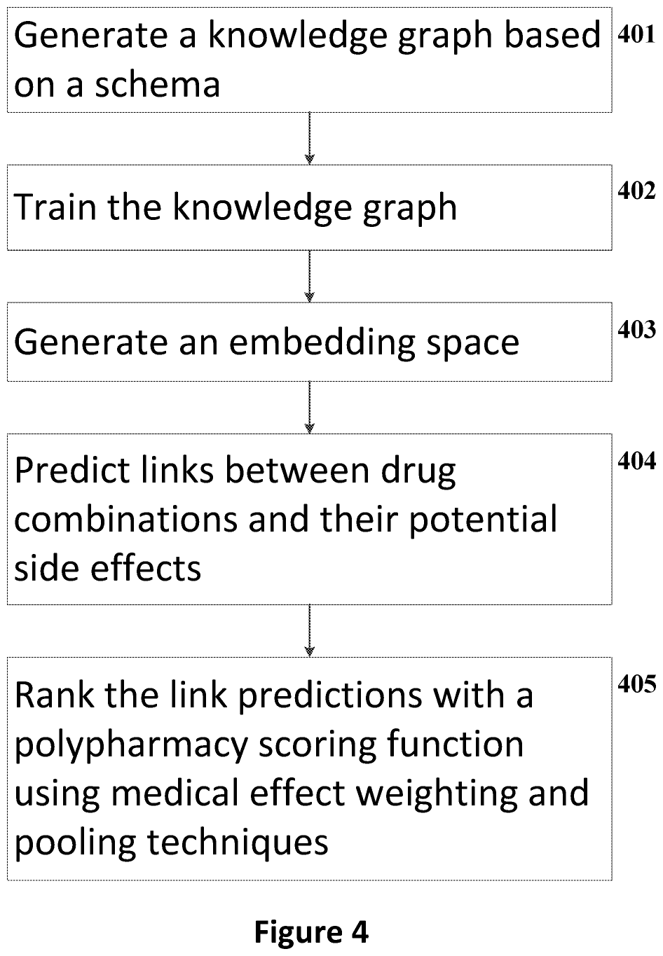

FIGS. 4-7 show flowcharts representing processes and methods, as implemented in accordance with certain embodiments. The processes may be implemented by a computing device, system, and/or circuitry components as described herein. FIG. 4 is a flowchart illustrating steps of an embodiment for ranking link predictions via the circuitry described in FIG. 2, in accordance with certain embodiments of the invention. The prediction rank may be obtained by the following process: generating the knowledge graph 2 based on the schema 3 (block 401); training the knowledge graph 2 (block 402); generating an embedding space 11 (block 403); predicting links or relationships 5 between drug combinations 9 and their potential side effects 10 (block 404); and, ranking the link predictions 15 with a polypharmacy scoring function 16 using medical effect weighting 18 and pooling techniques 24 (block 404).

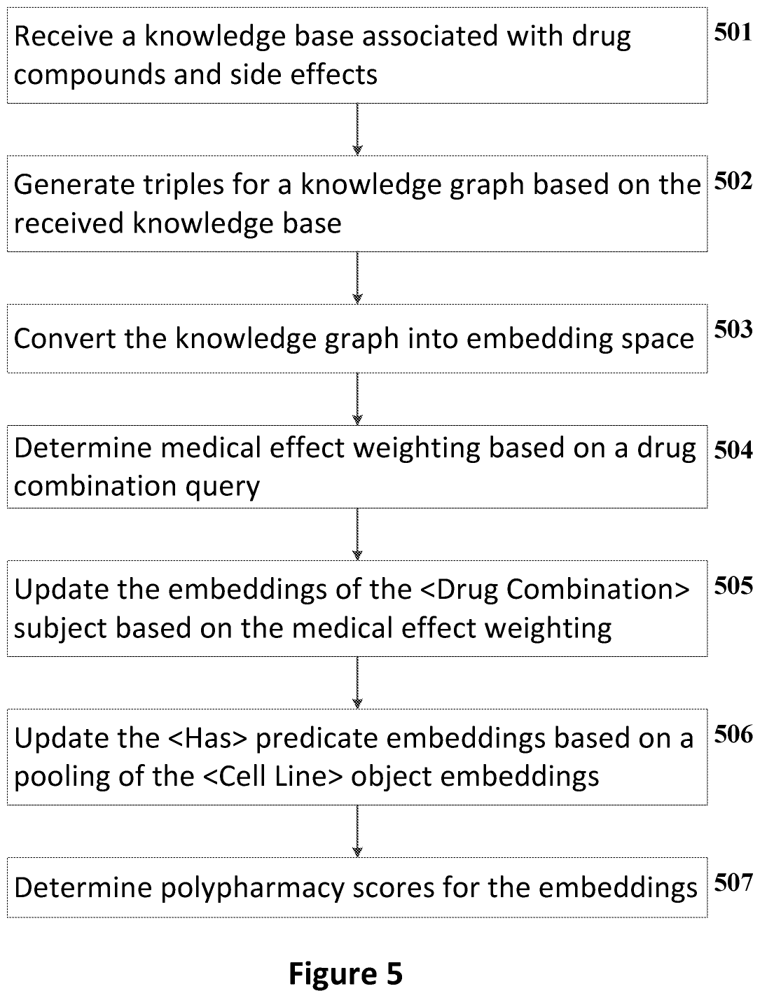

FIG. 5 is a diagram showing flowcharts representing steps in a process or method for determining link predictions and polypharmacy scores via the circuitry described in FIG. 2, in accordance with certain embodiments of the invention. At block 501, a knowledge base 1 is received. In embodiment, the knowledge base 1 may be associated with a plurality of drug compounds 8, drug combinations 9 and their associated side effects 10. In some embodiments, the received information may comprise an existing knowledge graph 2 with drug molecular-structure data 29 and drug side-effect data 30. In certain embodiments, the information received from the knowledge base 1 may be received from a combination index 19 of medical side effects for a combination of a plurality of drug compounds 8. Such a received knowledge base 1 may be training and enhanced by the knowledge graph generation circuitry 120. The knowledge graph generation circuitry 120 may construct a knowledge graph 2 based on the knowledge base 1. Triples 7 may be generated for a knowledge graph 2 based on the knowledge base 1 (block 502). The generated knowledge graph 2 may include nodes 4′ of information, and connecting edges 5′ representing a relationship 5 between nodes 4′ at a head-end of the edge 5′ and at a tail-end of the edge 4′. Referring back to FIG. 1, a schema 3 may be utilized by the knowledge graph generation circuitry 120 to generate the knowledge graph 2 with structured data within a scope of known drug compounds 8 and side effects 10.

The embedding space generation circuitry 130 may receive the knowledge graph 2 and convert it into an embedding space 11, as represented by block 503. The embedding space generation circuitry 130 may convert the structured data of the triples 7 from the knowledge graph 2 into embedding vectors 12. A vector 12 may comprise a metric representation of the triples 7 that have the following format: <head entity 4′, relationship 5′, tail entity 4′> (e.g., <Drug_Combination, has_Category, Side_Effect>). The vector conversion may be applied across the knowledge graph 2. The embedding space generation circuitry 130 may further implement the embedding space conversion by modeling the triples 7 according to an elaboration of a neural network architecture to learn the representations of the knowledge graph 2. In this way, the embedding space 11 may be constructed to comprise nodes 12′ (representing the embedding vectors 12) corresponding to the structured data comprising the knowledge graph 2. The nodes 12′ depicted in the embedding space 11 may correspond to drug combinations 9 that relate to a side effect 10 included in the knowledge graph 2.

The computation circuitry 140 may implement a polypharmacy score process. To begin, the computation circuitry 140 may initially receive a drug combination query 14 to identify drug formulations for at least two drug compounds 8 that are desired to be combined and further analyzed. The query 14 may further include desired compound attributes 6 for the new discovery node 5′ that will be determined for inclusion into the formulation. The computation circuitry 140 may determine a medical effect weighting 18 based on the drug combination query 14 (block 504). Various known methods may be used to determine the medical effect weighting 18. The method of calculating the medical effect weighting 18 of a drug combination 9 may comprise: a Loewe additivity score method, a Chou-Talalay combination index method, a Tau estimation method, a pharmacological independence method, and/or a Bliss statistical independence method. In an embodiment, the query 14 may be utilized to identify a medical effect weighting 18 using any of the above-mentioned methods.

The computation circuitry 140 may update the effects of a medical effect weighting 18 on the drug combinations 9 (block 505). In some embodiments, the embedding of the <Drug Combination> subject may be updated based on the medical effect weighting 18. The <Has> predicate embeddings 12 may be updated based on a pooling method 24 performed on the <Cell Line> object embeddings 12 (block 506). A polypharmacy score 20 may be determined for the new embeddings 12 that were updated based on the drug combination query 14, the medical effect weighting 18 and the pooling 24 (block 507).

In an embodiment, the polypharmacy scoring function 16 may utilize updated embeddings 12 of drug combination 9 based on a multiplication of a Loewe additivity score 18. The pooling 24 between the <Has> predicate and the <Cell Line> object may comprise element-wise multiplication the embeddings 12 of a side effect 10 and the embeddings 12 of the cell lines 25. From the calculated polypharmacy scores 20 for new links, the node 12′ with the highest discovery score 20 may be selected as the discovery node 12′, and the corresponding drug combination 9 may be selected for further experimental testing.

In an embodiment, the polypharmacy scoring function 16 may comprise an improvement of complex scoring function. The function may be based on the predicate embeddings 12 for a <Drug Combination> subject (e.g., A+B may represent the combination of drug A and drug B), embeddings 12 of a <Cell Line> subject (e.g., C1 may represent a cell line 25 for testing the drug combination 9) and embeddings 12 of an <Side Effect> object (e.g., E1 may represent a side effect 10 of the drug combination 9). In some embodiments, the medical effect weighting 18 of a drug combination 9 may be calculated based on the concentrations for two drug compounds 8 (e.g., a and b may represent the drug concentrations for drug A and drug B, respectively) and the effective dosages for the two compounds 8 (e.g., ED(A) and ED(B) may represent the effective dosages for drug A and drug B, respectively). In an embodiment, the medical effect weighting 18 may be calculated using the Loewe additivity score equation

$$f Loewe = l ( A + B ) = \frac{a}{E D ( A )} + \frac{b}{E D ( B )}$$

which may be multiplied with the drug combination 9,

l(A+B)(A+B))

in order to update the embeddings 12 of the drug combination 9. The predicate embeddings 12 for the cell lines 25 may be updated by pooling 24 between the linked relationships 5 and the cell line 25. Such pooling 24 may comprise element-wise multiplication the embeddings 12 of a side effect 10 and the embeddings 12 of the cell lines 25. A polypharmacy score 20 for new prediction links 15 may be calculated using the following equations:

fPolyPharm=Re(<l(A+B)(A+B),P(has,cell_line C1),side_effect E1>)

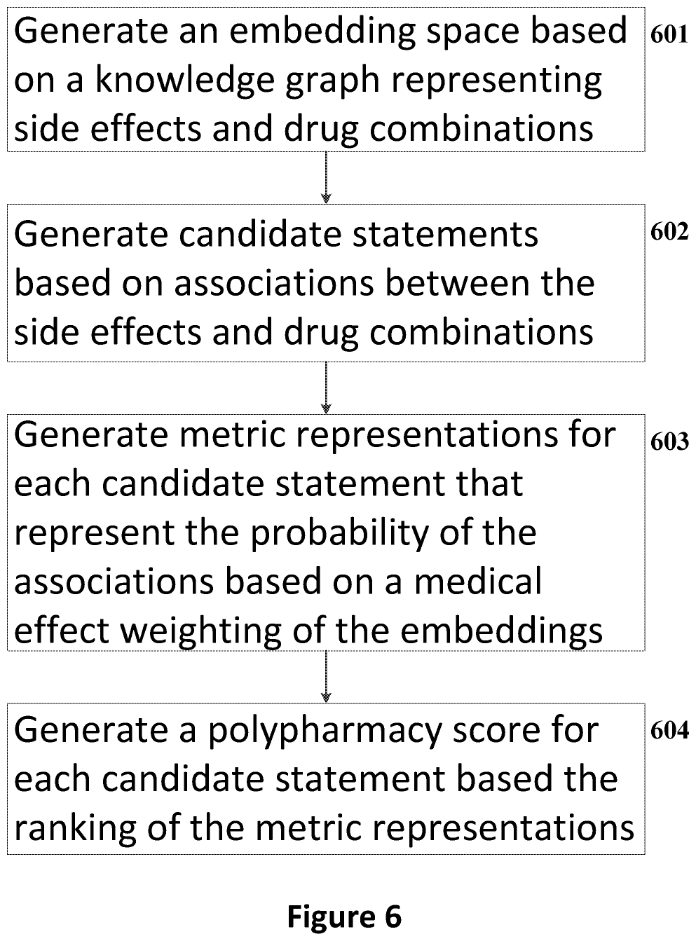

As shown in FIG. 6, an embodiment for the disclosed products, systems and methods may include the generation (block 601) of embeddings 12 based on knowledge graphs 2 that represent a knowledge base 1 of drug combinations 9 and side effects 10. The disclosed systems and methods may include the generation (block 602) of candidate statements 17 that may represent associations 27 between the side effects 10 and the drug combinations 9, and the generation (block 603) of metric representations 26 for each candidate statement 17. Each metric representation 26 may comprise the probability of an association 27 of a particular side effect 10 with a drug combination 9. The metric representations 26 may be generated using a medical effect weighting 18 of the embeddings 12. The medical effect weighting 18 may be based on a drug combination query 14. In an embodiment, the disclosed systems and methods may include the generation (block 604) of a polypharmacy score 20 for each candidate statement 17. The scores 20 may be based on the ranking 28 of the metric representations 26.

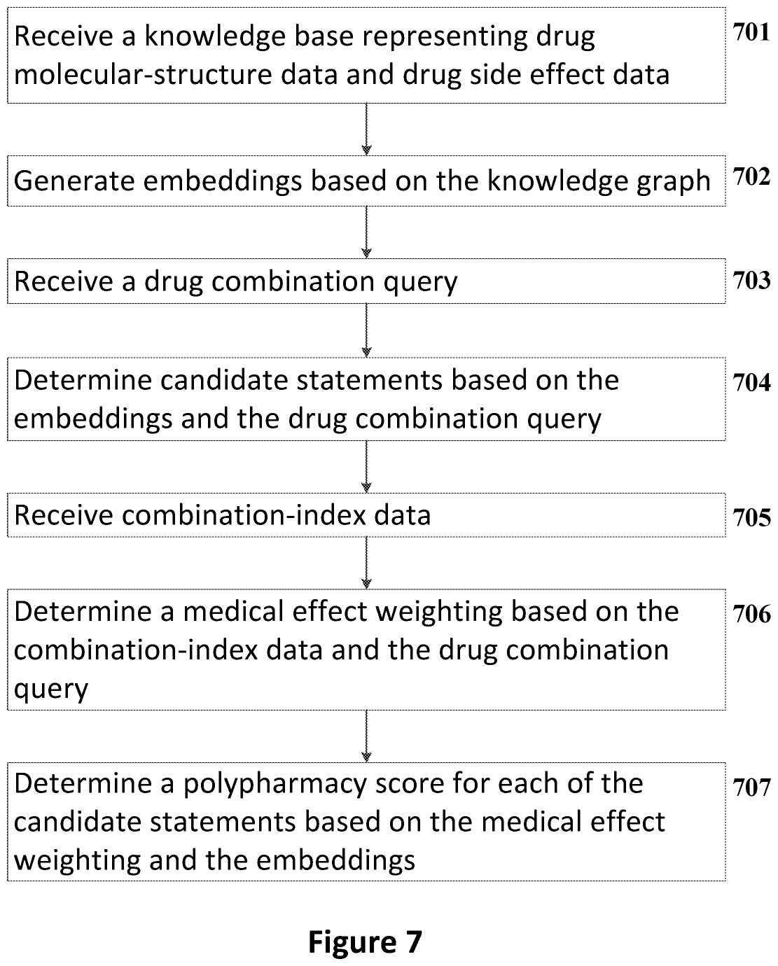

FIG. 7 illustrates another embodiment for the disclosed products, systems and methods. A knowledge graph 2 may be received (block 701) via a knowledge graph receiving circuitry 110. The knowledge graph 2 may represent drug molecular-structure data 29 and drug side-effect data 30. In certain embodiments, this data 29 and 30 may correspond to a plurality of drug combinations 9. The knowledge graph 2 may be based on a knowledge base 1. In some embodiments, the knowledge base 1 may comprise training data represented by an existing knowledge graph 2. The existing knowledge graph 2 may represent drug molecular-structure data 29 and drug side-effect data 30.

In an embodiment, the computation circuitry 140 may be configured to train the existing knowledge graph 2 and generate the knowledge graph 2 received by the knowledge graph receiving circuitry 110. In some embodiments, the knowledge graph 2 may be generated using a schema 3 providing node representations 4″ for drug compound data, chemical substructure data, drug combination data, gene data, cell line data, genotypic data, side-effect data, and disease classification data. The knowledge base 1 may comprise drug compound data, chemical substructure data, drug combination data, gene data, cell line data, genotypic data, side-effect data, and disease classification data.

At block 702, embeddings 12 may be generated based on the knowledge graph 2. The embeddings 12 may be generated via an embedding-space generation circuitry 130. A drug combination query 14 may be received (block 703) by a computation circuitry 140. A plurality of candidate statements 17 may be determined (block 704) based on the embeddings 12 and the drug combination query 14. The candidate statements 17 may represent associations 27 between a plurality of side effects 10 and a plurality of drug combinations 9. The candidate statements 17 may be determined via the computation circuitry 140.

In certain embodiments, combination-index data 31 may be received (block 705). The combination-index data 31 may represent medical effects of a combination 9 of drug compounds 8. The combination-index data 31 may be received by the computation circuitry 140. At block 706, a medical effect weighting 18 may be determined based on the combination-index data 31 and the drug combination query 14. The medical effect weighting 18 may be determined by the computation circuitry 140. The medical effect weighting 18 may be determined based on one of the following methods: a Loewe additivity score method, a Chou-Talalay combination index method, a Tau estimation method, a pharmacological independence method, and a Bliss statistical independence method.

A polypharmacy score 20 may be determined (block 707) for each of the candidate statements 17 based on the medical effect weighting 18 and the embeddings 12. Each polypharmacy score 20 may be a metric representation 26 comprising a probability of an association 27 between one of the side effects 10 and one of the drug combinations 9. The polypharmacy scores 20 may be determined via the computation circuitry 140. In some embodiments, the computation circuitry 140 may be configured to rank the metric representations 26 of the candidate statements 17. The computation circuitry 140 may be further configured to adjust the embeddings 12 based on a pooling 24 of the embeddings 12.

While the present disclosure has been particularly shown and described with reference to an embodiment thereof, it will be understood by those skilled in the art that various changes in form and details may be made therein without departing from the spirit and scope of the present disclosure. Although some of the drawings illustrate a number of operations in a particular order, operations that are not order-dependent may be reordered and other operations may be combined or broken out. While some reordering or other groupings are specifically mentioned, others will be apparent to those of ordinary skill in the art and so do not present an exhaustive list of alternatives.

What is claimed is:

1. A system for identifying side effects for drug combinations, comprising:
a knowledge graph receiver circuitry configured to receive a knowledge graph based on a knowledge base, the knowledge graph representing drug molecular-structure data and drug side-effect data;
an embedding-space generation circuitry configured to convert the knowledge graph into embeddings; and,
a computation circuitry configured to receive a drug combination query, the computation circuitry configured to determine a plurality of candidate statements based on the embeddings and the drug combination query, the candidate statements representing associations between a plurality of side effects and a plurality of drug combinations, the computation circuitry configured to receive combination-index data, the combination-index data representing medical effects of a combination of drugs, the computation circuitry configured to determine a medical effect weighting based on the combination-index data and the drug combination query, the computation circuitry configured to determine a polypharmacy score for each candidate statement based on the medical effect weighting and the embeddings, each polypharmacy score being a metric representation comprising a probability of an association between one of the side effects and one of the drug combinations.

2. The system of claim 1, wherein the knowledge base is an existing knowledge graph representing drug molecular-structure data and drug side-effect data.

3. The system of claim 1, wherein the knowledge base comprises drug compound data, chemical substructure data, drug combination data, gene data, cell line data, genotypic data, side-effect data, and disease classification data.

4. The system of claim 1, wherein the knowledge graph is generated using a schema providing node representations for drug compound data, chemical substructure data, drug combination data, gene data, cell line data, genotypic data, side-effect data, and disease classification data.

5. The system of claim 1, wherein the computation circuitry is further configured to rank the metric representations of the candidate statements.

6. The system of claim 1, wherein the computation circuitry is further configured to adjust the embeddings based on a pooling of the embeddings.

7. The system of claim 1, wherein the medical effect weighting is based on a method selected from a group consisting of: a Loewe additivity score method, a Chou-Talalay combination index method, a Tau estimation method, a pharmacological independence method, and a Bliss statistical independence method.

8. A method for identifying side effects for drug combinations, comprising:
receiving a knowledge graph representing drug molecular-structure data and drug side-effect data, wherein the knowledge graph is based on a knowledge base, wherein the knowledge graph is received via a knowledge graph receiving circuitry;
generating embeddings based on the knowledge graph, wherein the embeddings are generated via an embedding-space generation circuitry;
receiving a drug combination query, wherein the drug combination query is received via a computation circuitry;
determining a plurality of candidate statements based on the embeddings and the drug combination query, wherein the candidate statements represent associations between a plurality of side effects and a plurality of drug combinations, wherein the candidate statements are determined via the computation circuitry;
receiving combination-index data, wherein the combination-index data represents medical effects of a combination of drugs, wherein the combination-index data is received via the computation circuitry;
determining a medical effect weighting based on the combination-index data and the drug combination query, wherein the medical effect weighting is determined via the computation circuitry; and,
determining a polypharmacy score for each of the candidate statements based on the medical effect weighting and the embeddings, wherein each polypharmacy score is a metric representation comprising a probability of an association between one of the side effects and one of the drug combinations, wherein the polypharmacy scores are determined via the computation circuitry.

9. The method of claim 8, wherein the knowledge base is an existing knowledge graph representing drug molecular-structure data and drug side-effect data.

10. The method of claim 8, wherein the knowledge base comprises drug compound data, chemical substructure data, drug combination data, gene data, cell line data, genotypic data, side-effect data, and disease classification data.

11. The method of claim 8, wherein the knowledge graph is generated using a schema providing node representations for drug compound data, chemical substructure data, drug combination data, gene data, cell line data, genotypic data, side-effect data, and disease classification data.

12. The method of claim 8, wherein the computation circuitry is further configured to rank the metric representations of the candidate statements.

13. The method of claim 8, wherein the computation circuitry is further configured to adjust the embeddings based on a pooling of the embeddings.

14. The method of claim 8, wherein the medical effect weighting is based on a method selected from a group consisting of: a Loewe additivity score method, a Chou-Talalay combination index method, a Tau estimation method, a pharmacological independence method, and a Bliss statistical independence method.

15. A product for identifying side effects for drug combinations, comprising:
a machine-readable medium, other than a transitory signal; and,
instructions stored on the machine-readable medium, the instructions configured to, when executed, cause processing circuitry to:
receive a knowledge graph representing drug molecular-structure data and drug side-effect data, wherein the knowledge graph is based on a knowledge base, wherein the knowledge graph is received via a knowledge graph receiving circuitry;
generate embeddings based on the knowledge graph, wherein the embeddings are generated via an embedding-space generation circuitry;
receive a drug combination query, wherein the drug combination query is received via a computation circuitry;
determine a plurality of candidate statements based on the embeddings and the drug combination query, wherein the candidate statements represent associations between a plurality of side effects and a plurality of drug combinations, wherein the candidate statements are determined via the computation circuitry;
receive combination-index data, wherein the combination-index data represents medical effects of a combination of drugs, wherein the combination-index data is received via the computation circuitry;
determine a medical effect weighting based on the combination-index data and the drug combination query, wherein the medical effect weighting is determined via the computation circuitry; and,
determine a polypharmacy score for each of the candidate statements based on the medical effect weighting and the embeddings, wherein each polypharmacy score is a metric representation comprising a probability of an association between one of the side effects and one of the drug combinations, wherein the polypharmacy scores are determined via the computation circuitry.

16. The product of claim 15, wherein the knowledge base is an existing knowledge graph representing drug molecular-structure data and drug side-effect data.

17. The product of claim 15, wherein the knowledge base comprises drug compound data, chemical substructure data, drug combination data, gene data, cell line data, genotypic data, side-effect data, and disease classification data.

18. The product of claim 15, wherein the knowledge graph is generated using a schema providing node representations for drug compound data, chemical substructure data, drug combination data, gene data, cell line data, genotypic data, side-effect data, and disease classification data.

19. The product of claim 15, wherein the computation circuitry is further configured to rank the metric representations of the candidate statements.

20. The product of claim 15, wherein the computation circuitry is further configured to adjust the embeddings based on a pooling of the embeddings.

21. The product of claim 15, wherein the medical effect weighting is based on a method selected from a group consisting of: a Loewe additivity score method, a Chou-Talalay combination index method, a Tau estimation method, a pharmacological independence method, and a Bliss statistical independence method.
